(19) United States
(12) Patent Application Publication
(10) Pub. No.: US 2009/0204203 A1
(43) Pub. Date: Aug. 13, 2009

(54) Bioabsorbable Stent Having a Radiopaque Marker

(75) Inventors: Jeffrey Allen, Santa Rosa, CA (US); Matthew J. Birdsall, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/027,715

(22) Filed: Feb. 7, 2008

(51) Int. Cl. A61F 2/82

(52) U.S. Cl. 623/1.34; 623/1.38

(57) ABSTRACT

A bioabsorbable stent includes one or more radiopaque markers. The stent body may include a generally cylindrical body portion and a marker support for receiving the one or more marker(s). The marker support may be connected to an end of the body portion, or may be an integral portion of the body portion. By selectively controlling dissolution of the biodegradable material of the marker support, the marker support will remain intact for a sufficient time to allow for the marker to endothelialize and therefore prevent the marker from dislodging and embolizing. The controlled dissolution may be accomplished via one or more of the following mechanisms, including increasing the cross-sectional thickness of the marker support, passivating or oxidizing the marker support, utilizing a different, slower absorbing material for the marker support, utilizing a bioabsorbable polymeric coating on the marker support, or protecting the marker support with a sacrificial anode.

### FIELD OF THE INVENTION

The invention relates generally to temporary endoluminal prostheses for placement in a body lumen, and more particularly to stents that are bioabsorbable.

### BACKGROUND OF THE INVENTION

A wide range of medical treatments exist that utilize “endoluminal prostheses.” As used herein, endoluminal prostheses is intended to cover medical devices that are adapted for temporary or permanent implantation within a body lumen, including both naturally occurring and artificially made lumens, such as without limitation: arteries, whether located within the coronary, mesentery, peripheral, or cerebral vasculature; veins; gastrointestinal tract; biliary tract; urethra; trachea; hepatic shunts; and fallopian tubes.

Accordingly, a wide assortment of endoluminal prostheses have been developed, each providing a uniquely beneficial structure to modify the mechanics of the targeted lumen wall. For example, stent prostheses are known for implantation within body lumens to provide artificial radial support to the wall tissue, which forms the various lumens within the body, and often more specifically, for implantation within the blood vessels of the body.

Essentially, stents that are presently utilized are made to be permanently or temporarily implanted. A permanent stent is designed to be maintained in a body lumen for an indeterminate amount of time and is typically designed to provide long term support for damaged or traumatized wall tissues of the lumen. There are numerous conventional applications for permanent stents including cardiovascular, urological, gastrointestinal, and gynecological applications. A temporary stent is designed to be maintained in a body lumen for a limited period of time in order to maintain the patency of the body lumen, for example, after trauma to a lumen caused by a surgical procedure or an injury.

Permanent stents, over time, may become encapsulated and covered with endothelium tissues, for example, in cardiovascular applications, causing irritation to the surrounding tissue. Further, if an additional interventional procedure is ever warranted, a previously permanently implanted stent may make it more difficult to perform the subsequent procedure.

Temporary stents, on the other hand, preferably do not become incorporated into the walls of the lumen by tissue ingrowth or encapsulation. Temporary stents may advantageously be eliminated from body lumens after an appropriate period of time, for example, after the traumatized tissues of the lumen have healed and a stent is no longer needed to maintain the patency of the lumen. As such, temporary stents may be removed surgically or be made bioabsorbable/biodegradable.

Temporary stents may be made from bioabsorbable and/or biodegradable materials that are selected to absorb or degrade in vivo over time. However, there are disadvantages and limitations associated with the use of bioabsorbable or biodegradable stents. Limitations arise in controlling the breakdown of the bioabsorbable materials from which such stents are made, as in, preventing the material from breaking down too quickly or too slowly. If the material is absorbed too quickly, the stent will not provide sufficient time for the vessel to heal, or if absorbed too slowly, the attendant disadvantages of permanently implanted stents may arise.

There is a need for a temporary stent that provides sufficient support in a body lumen for the duration of a therapeutically appropriate period of time, which then degrades to be eliminated from the patient's body without surgical intervention. Magnesium appears to be a suitable material for providing both strength and bioabsorbability to a stent. A magnesium stent may handle like an ordinary metallic stent, because it plastically deforms and thus have limited recoil, but also may be engineered so as to be absorbable within the body. That is, such a magnesium stent has all the good handling characteristics of a non-biodegradable metal stent while still providing an absorbable stent platform. Such magnesium stents, however, are not very radiopaque because magnesium does not show up well under fluoroscopy. Accordingly, it would be beneficial if such a magnesium bioabsorbable stent could be made to be more radiopaque or visible under a fluoroscopic device.

It is known to utilize a radiopaque marker with an ordinary metallic stent to make the stent more visible under a fluoroscopic device. However, a problem that arises using a radiopaque marker with a biodegradable stent is a risk of embolism caused by the dislodgement of the marker that can then move downstream, which may occur when the biodegradable stent is absorbed by the body, but the marker is not. Once the stent biodegrades, the marker may embolize and block the coronary arteries, or migrate further downstream, causing additional complications. Thus, it would be beneficial if such a bioabsorbable stent could be made to be more radiopaque without increasing the risk of embolism caused by the dislodgement of the marker.

### BRIEF SUMMARY OF THE INVENTION

Embodiments of the present invention are directed to an intraluminal stent device. In one embodiment of the invention, the stent has a biodegradable body portion having a proximal end, a distal end, and a generally cylindrical hollow shape. The body portion has a first thickness. The stent also includes at least one biodegradable marker support having a second thickness and a radiopaque marker attached to the marker support. The second thickness is greater than the first thickness so that upon implantation of the stent within the vasculature, dissolution of the marker support is selectively controlled to biodegrade slower than the remaining body portion of the stent in order to allow the marker to endothelialize. The body portion and marker support may be formed of magnesium or a magnesium alloy, and the radiopaque marker may be formed from tantalum.

In another embodiment of the invention, the stent has a biodegradable body portion having a proximal end, a distal end, and a generally cylindrical hollow shape. The stent includes at least one biodegradable marker support and a radiopaque marker attached to the marker support. A bioabsorbable coating is placed over at least a portion of the marker support so that upon implantation of the stent within the vasculature dissolution of the marker support is selectively controlled to biodegrade slower than the remaining body portion of the stent in order to allow the marker to endothelialize.

In another embodiment of the invention, the stent has a biodegradable body portion having a proximal end, a distal end, and a generally cylindrical hollow shape. The stent includes at least one biodegradable marker support and a radiopaque marker attached to the marker support. A corrosion-resistant layer is formed by oxidizing or passivating at least a portion of the marker support so that upon implantation of the stent within the vasculature dissolution of the marker support is selectively controlled to biodegrade slower than the remaining body portion of the stent in order to allow the marker to endothelialize.

In another embodiment of the invention, the stent has a biodegradable body portion having a proximal end, a distal end, and a generally cylindrical hollow shape. The stent also includes at least one biodegradable marker support formed of a first biodegradable material having a first corrosion potential and a radiopaque marker attached to the marker support. A sacrificial anode is electrically connected to the marker support, wherein the sacrificial anode is formed of a second biodegradable material having a second corrosion potential that is higher than the first corrosion potential of the marker support so that upon implantation of the stent within the vasculature dissolution of the marker support is selectively controlled to biodegrade slower than the body portion of the stent in order to allow the marker to endothelialize.

In another embodiment of the invention, the stent has a biodegradable body portion having a proximal end, a distal end, and a generally cylindrical hollow shape. The body portion is formed of a first biodegradable material having a first dissolution rate. The stent also includes at least one biodegradable marker support formed of a second biodegradable material having a second dissolution rate and a radiopaque marker attached to the marker support. The second dissolution rate is slower than the first dissolution rate so that upon implantation of the stent within the vasculature dissolution of the marker support is selectively controlled to biodegrade slower than the remaining body portion of the stent in order to allow the marker to endothelialize.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 a perspective view of an exemplary stent in accordance with an embodiment of the present invention.

FIG. 2 is a plan view of a flattened stent strut in accordance with an embodiment of the present invention.

FIG. 3 is a plan view of a marker assembly in accordance with an embodiment of the present invention.

FIG. 4 is a cross-sectional view of the marker assembly taken along line C-C of FIG. 3.

FIG. 5 is a cross-sectional view of the marker assembly taken along line D-D of FIG. 3.

FIG. 6 is a cross-sectional view of a stent strut taken along line A-A of FIG. 1.

FIG. 7 is a cross-sectional view of a marker support of the marker assembly taken along line B-B of FIG. 2.

FIG. 8 is a plan view of a flattened stent strut in accordance with another embodiment of the present invention.

FIG. 9 is a cross-sectional view of a marker support of the marker assembly taken along line B-B of FIG. 2 in accordance with another embodiment of the present invention.

FIG. 10 is a cross-sectional view of a marker support of the marker assembly taken along line B-B of FIG. 2 in accordance with another embodiment of the present invention.

FIG. 11 is a cross-sectional view of the marker assembly taken along line C-C of FIG. 3 in accordance with another embodiment of the present invention.

FIG. 12 is a cross-sectional view of a marker support of the marker assembly taken along line B-B of FIG. 2 in accordance with another embodiment of the present invention.

FIG. 13 is a plan view of a flattened stent strut in accordance with another embodiment of the present invention.

FIG. 14 is a side elevational view of a stent delivery system in accordance with an embodiment of the present invention.

FIG. 15 is a plan view of a flattened stent strut in accordance with another embodiment of the present invention.

FIG. 16A is a cross-sectional view of the stent strut taken along line A-A of FIG. 15 in accordance with an embodiment of the present invention.

FIG. 16B is a cross-sectional view of the stent strut taken along line A-A of FIG. 15 in accordance with another embodiment of the present invention.

FIG. 17 is a plan view of a flattened stent strut having a marker assembly in accordance with another embodiment of the present invention.

FIG. 18A is a cross-sectional view of the marker assembly taken along line A-A of FIG. 17 in accordance with an embodiment of the present invention.

FIG. 18B is a cross-sectional view of the marker assembly taken along line A-A of FIG. 17 in accordance with another embodiment of the present invention.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. Although the description of the invention is in the context of treatment of blood vessels such as the coronary, carotid and renal arteries, the invention may also be used in any other body passageways where it is deemed useful. Furthermore, there is no intention to be bound by any expressed or implied theory presented in the preceding technical field, background, brief summary or the following detailed description.

Embodiments of the present invention relate to a bioabsorbable stent having one or more radiopaque markers that are visible to a physician viewing, for example, an X-ray fluoroscopy device while deploying and/or positioning the stent into the body vessel. Radiopaque markers are generally secured to the proximal and/or distal ends of the stent extending outwardly from one or more peaks or troughs of undulating bands of the stent body. Embodiments of the present invention are directed to underlying stent structures that allow the one or more markers to endothelialize. Because the stent bioresorbs or breaks down and the marker does not, it is important that the marker remains fixed and stable during bioresorption of the stent body. By controlling dissolution of an area of the stent near the marker, the marker may endothelialize and is therefore prevented from dislodging and embolizing. Thus, the bioabsorbable stent may be made more radiopaque without increasing the risk of embolism caused by the dislodgement of the marker.

Dissolution of the biodegradable stent material or portion holding the marker in place (hereinafter referred to as marker support) is controlled or slowed so that it will remain intact for a sufficient time to allow for marker endothelialization. The term “endothelialization” is meant to describe the process in which a foreign object, such as the marker in embodiments of the present invention, becomes incorporated into the walls of the lumen by tissue ingrowth or encapsulation. Thus, in other words, dissolution of the marker support is controlled so that the marker is held against the vessel wall long enough to endothelialize. As part of the vessel wall, the marker is stable and will not migrate downstream and thus avoids causing potential complications. Dissolution of the marker support must be controlled or slowed for a sufficient time to allow for endothelialization to occur, approximately three to six weeks. The biodegradable body portion of the stent has a first dissolution rate and the marker support has a second dissolution rate. The second dissolution rate is slower than the first dissolution rate. In particular, the second dissolution rate is approximately 30-100% slower than the first dissolution rate in order to allow the radiopaque marker to endothelialize. The controlled dissolution of the marker support may be accomplished via one or more mechanisms that include the following: increasing the cross-sectional thickness of the marker support, passivating or oxidizing the marker support, utilizing a different, slower absorbing material for marker support, utilizing a bioabsorbable polymeric coating on the marker support, anodically protecting the marker support with a sacrificial anode, and any other suitable means of slowing absorption or corrosion in the region that secures the marker.

In an embodiment of the present invention, rather than delay dissolution of the entire stent in order to allow the radiopaque marker to endothelialize, it is desirable to selectively control or delay dissolution of only the stent material securing the marker. Selectively controlling dissolution of only the marker support material allows the remainder of the stent body to be absorbed in a desired amount of time and avoids the risk of the stent body becoming encapsulated and covered with endothelium tissues. In other words, if dissolution of the entire stent was controlled or delayed in order to allow the radiopaque marker to endothelialize, the stent body may also endothelialize and thus may not break down as desired. The biodegradable stent body must be in contact with a body fluid such as blood in order for the stent to corrode or be absorbed into the body as desired. Thus, selectively controlling dissolution of only the marker support avoids the undesirable endothelialization of the stent body.

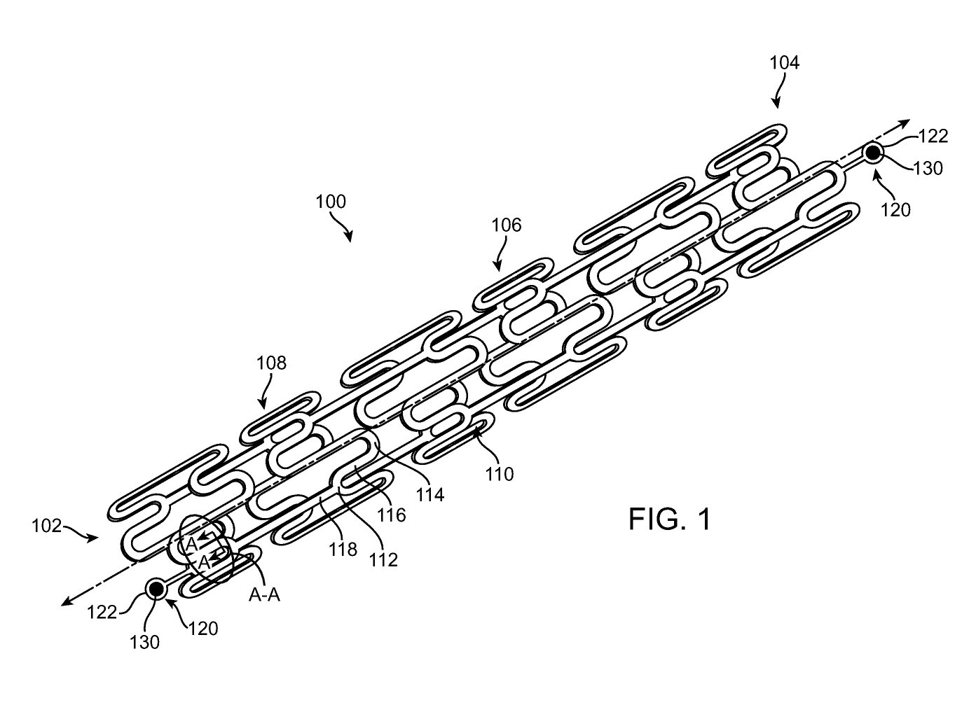

In one embodiment of the present invention, the biodegradable stent is formed of magnesium or a magnesium alloy and the marker is formed of tantalum. However, the marker may be formed of any other relatively heavy metal which is generally visible by X-ray fluoroscopy such as tantalum, titanium, platinum, gold, silver, palladium, iridium, and the like. In addition, the stent may be formed of any suitable biodegradable or bioabsorbable material, including metals and polymers. Further details and description of the embodiments of the present invention are provided below with reference to FIGS. 1-18B.

FIG. 1 illustrates an endoluminal prosthesis in accordance with an embodiment of the present invention. Stent 100 includes a generally cylindrical hollow body portion 106 extending between a proximal end 102 and a distal end 104. Body portion 106 is configured to fit into a body lumen such as a blood vessel. Stent 100 also includes at least one marker assembly 120 which may be located at one or both ends of body portion 106. For example, in FIG. 1, marker assembly 120 is shown at proximal end 102 and at distal end 104. Marker assembly 120 includes a marker support 122 and a marker 130 that is formed of a radiopaque material which is visible to a physician viewing, for example, an X-ray fluoroscopy device while deploying and/or positioning stent 100 into the target body vessel, as described in detail below.

According to embodiments of the present invention, body portion 106 may have a generally tubular or cylindrical expandable structure and may be circularly symmetric with respect to a central longitudinal axis. Stent 100 is a patterned tubular device that includes a plurality of radially expandable cylindrical rings 108 aligned on a common longitudinal axis to form a generally cylindrical hollow body having a radial and longitudinal axis. Cylindrical rings 108 may be formed from struts 110 having a generally sinusoidal pattern including peaks 112, valleys 114, and generally straight segments 116 connecting peaks 112 and valleys 114. Connecting links 118 connect adjacent cylindrical rings 108 together. In FIG. 1, connecting links 118 are shown as generally straight links connecting peak 112 of one ring 108 to valley 114 of an adjacent ring 108. However, connecting links 118 may connect a peak 112 of one ring 108 to a peak 112 of an adjacent ring, or a valley 114 to a valley 114, or a straight segment 116 to a straight segment 116. Further, connecting links 118 may be curved. Connecting links 118 may also be excluded, with a peak 112 of one ring 108 being directly attached to a valley 114 of an adjacent ring 108, such as by welding, soldering, or the manner in which stent 100 is formed, such as by etching the pattern from a flat sheet or a tube. An outer diameter of body portion 106 may be approximately equal to or slightly larger than an inner diameter of a target body vessel and may be substantially constant along the central longitudinal axis.

It will be appreciated by one of ordinary skill in the art that stent 100 of FIG. 1 is merely an exemplary stent and that stents of various forms and methods of fabrication can be used in accordance with various embodiments of the present invention. For example, in a typical method of making a stent, a thin-walled, small diameter metallic tube is cut to produce the desired stent pattern, using methods such as laser cutting or chemical etching. The cut stent may then be de-scaled, polished, cleaned and rinsed. Some examples of methods of forming stents and structures for stents are shown in U.S. Pat. No. 4,733,665 to Palmaz, U.S. Pat. No. 4,800,882 to Gianturco, U.S. Pat. No. 4,886,062 to Wiktor, U.S. Pat. No. 5,133,732 to Wiktor, U.S. Pat. No. 5,292,331 to Boneau, U.S. Pat. No. 5,421,955 to Lau, U.S. Pat. No. 5,935,162 to Dang, U.S. Pat. No. 6,090,127 to Globerman, and U.S. Pat. No. 6,730,116 to Wolinsky et al., each of which is incorporated by reference herein in its entirety. Further, balloon-expandable stents may also be utilized in various embodiments of the present invention, such as those disclosed in U.S. Pat. No. 5,776,161 to Globerman, U.S. Pat. No. 6,113,627 to Jang, and U.S. Pat. No. 6,663,661 to Boneau, each of which is incorporated by reference herein in its entirety.

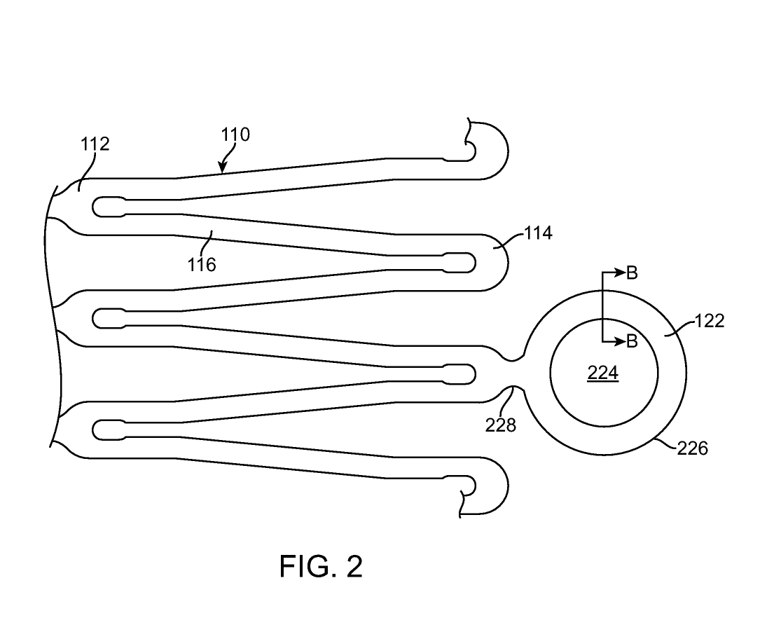

FIG. 2 shows a single stent strut 110 in accordance with an embodiment of the present invention. Stent strut 110 is shown as if the generally cylindrical ring 108 has been cut and the stent strut 110 has been laid out flat. Marker support 122 is shown attached to stent strut 110 via connection 228. In this embodiment, marker support 122 is an annular or ring shape, having an inner or interior volume 224 and an outer or peripheral surface 226. Radiopaque marker 130 (not shown in FIG. 2) is received or located within inner volume 224 of the annular or ring shaped marker support 122. Marker support 122 may have any suitable shape, including circular or rectangular, as long as it is adapted to receive radiopaque marker 130. Connection 228 may be formed by welding stent strut 110 to marker support 122 such as by resistance welding, friction welding, laser welding or another form of welding such that no additional materials are used to connect stent strut 110 and marker support 122. Alternatively, stent strut 110 and marker support 122 can be connected by soldering, by the addition of a connecting element there between, or by another mechanical method. Further, stent strut 110 and marker support 122 may be formed pre-connected as a unitary structure, such as by laser cutting or etching the entire stent body from a hollow tube or sheet. Other connections or ways to connect stent strut 110 and marker support 122 would be apparent to one skilled in the art and are included herein. To describe the particular structure of stent 100, stent strut 110 and marker support 122 may be described as being connected or coupled to each other. Thus, the terms “connect with,” “connected,” or “coupled” may mean either naturally continuing (or flowing together) or mechanically coupled together.

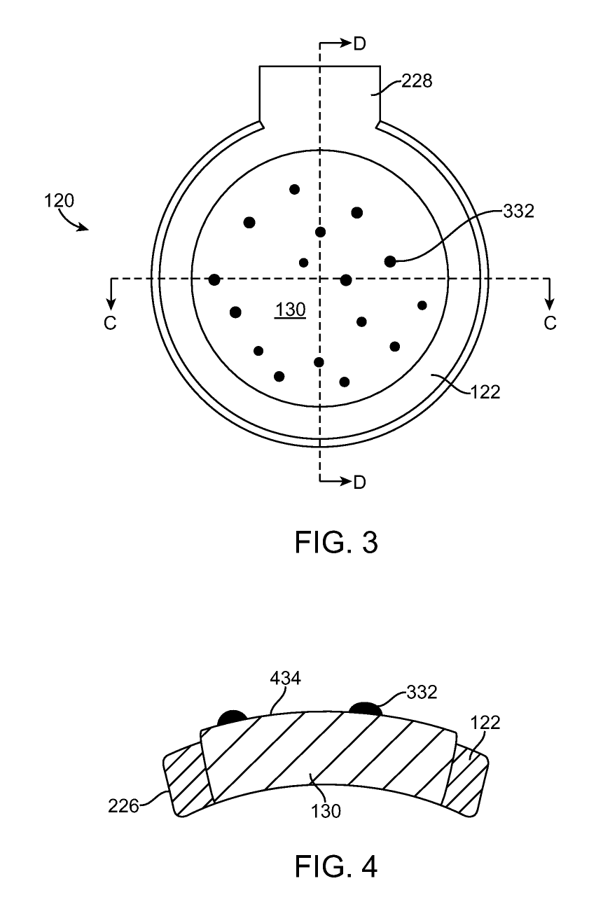

FIG. 3 is a plan view of marker assembly 120 including radiopaque marker 130 received or located within inner volume 224 of the annular or ring shaped marker support 122. In order to ensure marker 130 is not dislodged from the stent body, marker 130 must remain securely fixed to marker support 122 during delivery and deployment. Securement may be accomplished via various mechanisms, including press fitting, diffusion bonding, crimping, and/or metallization coating.

Due to the respective materials of marker support 122 and marker 130, marker support 122 bioresorbs or dissociates in vivo and marker 130 does not. More particularly, body portion 106 of stent 100 (including stent strut 110 and marker support 122) is constructed from a biodegradable or bioabsorbable material. In one embodiment, body portion 106 is constructed out of magnesium or a magnesium alloy, including formulations that have approximately 50-98% magnesium. A bioabsorbable metal is preferred because of its greater structural strength. Alternatively, body portion 106 can be formed of a suitable biodegradable or bioabsorbable polymer material, such as polyactic acid, polyglycolic acid, collagen, polycaprolactone, hylauric acid, co-polymers of these materials, as well as composites and combinations thereof.

Marker 130 is formed of a radiopaque material that is visible to a physician viewing, for example, an X-ray fluoroscopy device while deploying and/or positioning stent 100 into the target body vessel. In one embodiment, marker 130 is formed of tantalum. However, marker 130 may be formed from any suitable biocompatible material that enhances the radiopacity of stent 100, including tantalum, titanium, platinum, gold, silver, palladium, iridium, zirconium, barium, bismuth, and iodine.

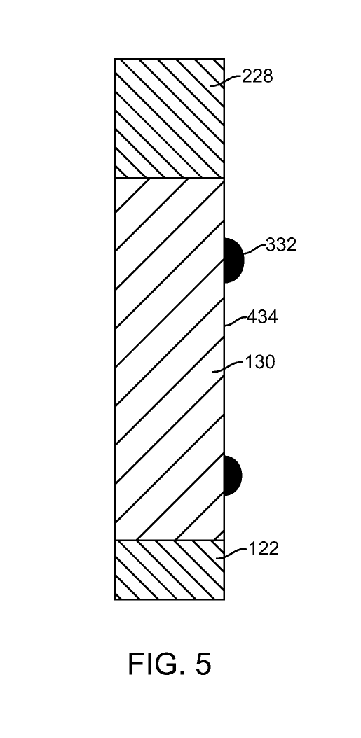

In one embodiment of the present invention, as shown in FIGS. 3-5, marker 130 may include protrusions 332 on an outer surface 434 of marker 130 in order to facilitate endothelialization of marker 130. FIG. 4 is a cross-sectional view of marker assembly 120 taken along line C-C of FIG. 3, and FIG. 5 is a cross-sectional view of marker assembly 120 taken along line D-D of FIG. 3. Protrusions 332 impart an irregular surface to the marker 130, thereby enhancing friction between outer surface 434 of marker 130 and the vessel wall. The irregular surface provided by protrusions 332 provides ingrowth sites for fibrotic tissue for retaining the marker 130 in place after implantation. Protrusions 332 may have any suitable shape or configuration, for example, including round, circular bumps as shown in FIGS. 3-5. Another configuration for protrusions 332 may be rectangular ribs. Further, other configurations may be utilized for imparting an irregular surface onto marker 130 such as indentations formed on outer surface 434 of marker 130. Each of these structures would provide sites for fibrotic tissue growth for marker retention. Outer surface 434 of marker 130 may be curved as shown in FIG. 4 in order to facilitate conformance to the vessel wall.

In another embodiment of the present invention, marker 130 may be relatively porous in order to facilitate endothelialization of marker 130. For example, marker 130 may include a porous, tissue-engaging outer surface which promotes rapid tissue ingrowth and consequent marker stabilization. The porous surface may be formed by sintering or otherwise adhering small particles of metal or other granulated material to the outer surface of marker 130. The sintered metallic material may be the same material as that forming marker 130, or may be a different material. The porous surface may also be formed by dealloying and/or chemical etching processes known in the art. A relatively porous outer surface facilitates migration of cells (e.g. fibroblasts and endothelial cells) into and through marker 130 such that marker 130 may become incorporated into the walls of the lumen by tissue ingrowth.

As previously stated, due to the respective materials of body portion 106 and marker 130, body portion 106 (including stent struts 110 and marker support 122) bioresorbes or dissociates in vivo and marker 130 does not. It is desirable to assure that marker 130 remains fixed and stable during bioresorption of body portion 106. Embodiments of the present invention are directed to selectively controlling dissolution of marker support 122 so that marker 130 may endothelialize and therefore be prevented from dislodging and embolizing. Particularly dissolution of the biodegradable material of marker support 122 is controlled or slowed so that marker support 122 will remain intact a sufficient time to allow for marker 130 to endothelialize, for example, three to six weeks. Thus, stent 100 may be made more radiopaque by the inclusion of marker 130 without increasing the risk of embolism. The controlled dissolution may be accomplished via one or more of the following mechanisms discussed in more detail below, including increasing the cross-sectional thickness of marker support 122 relative to the cross-sectional thickness of stent strut 110, utilizing a different, slower absorbing material for marker support 122 relative to stent strut 110, passivating or oxidizing marker support 122, utilizing a bioabsorbable polymeric coating on marker support 122, anodically protecting marker support 122 with a sacrificial anode, or any other suitable means of slowing absorption or corrosion of marker support 122.

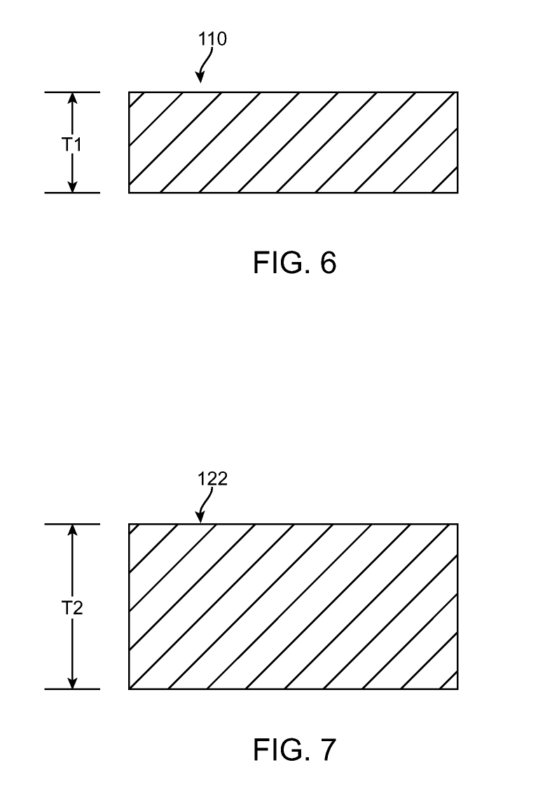

In one embodiment, the dissolution control mechanism is increasing the cross-sectional thickness of marker support 122 relative to the cross-sectional thickness of stent strut 110, as shown in FIGS. 6-7. FIG. 6 is a cross-sectional view of stent strut 110 taken along line A-A of FIG. 1, while FIG. 7 is a cross-sectional view of marker support 122 taken along line B-B of FIG. 2. As visible through a comparison of FIGS. 6-7, stent strut 110 has a thickness T1 and marker support 122 has a thickness T2, wherein T2 is greater than T1. Marker support 122 will thus bioresorb or break down in vivo slower than stent strut 110 due to the increase in the amount of material at marker support 122. Accordingly, dissolution of the biodegradable material of marker support 122 is controlled or slowed so that marker support 122 will remain intact for a sufficient time to allow for marker 130 to endothelialize. Thickness T2 may be selected such that the dissolution of marker support 122 takes approximately three to six weeks and thus allows marker 130 to endothelialize.

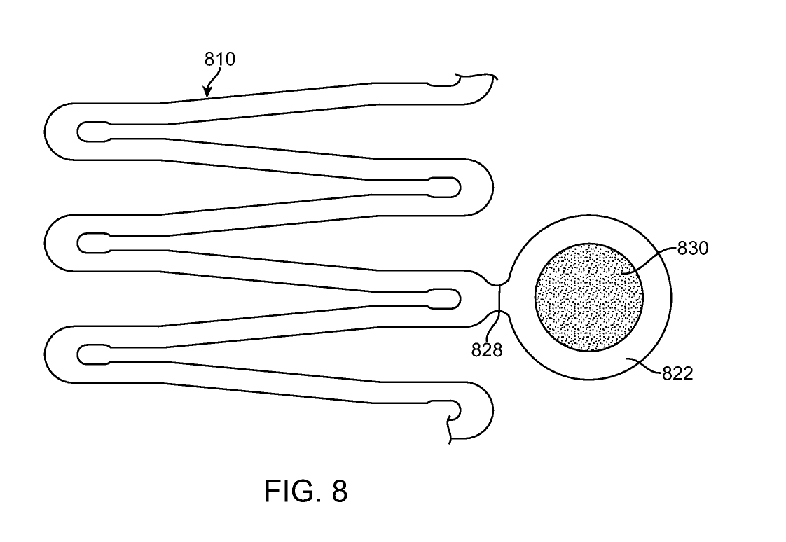

In another embodiment of the present invention illustrated in FIG. 8, the dissolution control mechanism is forming marker support 822 and stent strut 810 from different biodegradable materials. More particularly, stent strut 810 is formed from a first biodegradable or bioabsorbable material having a first dissolution rate. Marker support 822 is formed from a second biodegradable or bioabsorbable material having a second dissolution rate. The second dissolution rate is slower than the first dissolution rate so that marker support 822 may remain intact for a sufficient time for marker 830 to endothelialize. Each type of bioabsorbable or biodegradable material has a characteristic degradation rate in the body. Some materials are relatively fast-bioabsorbing materials (weeks to months) while others are relatively slow-bioabsorbing materials (months to years). By forming marker support 822 of a different, slower absorbing material than stent strut 810, the majority of the stent body will bioresorb or break down relatively quickly while marker support 822 remains intact for a sufficient time for marker 830 to endothelialize. For example, the stent strut 810 may be constructed out of magnesium or a magnesium alloy, having a high percentage of magnesium. Marker support 822 may also be constructed of a magnesium alloy. However, the alloy chemistry of marker support 822 may be varied to produce a more noble alloy having a slower dissolution rate, such as a combination of magnesium (Mg) alloyed with iron (Fe). Marker support 822 is shown attached to stent strut 810 via connection 828. For example, marker support 822 may be welded or otherwise mechanically attached to stent strut 810.

In another embodiment of the present invention, the dissolution control mechanism is utilizing a bioabsorbable coating on marker support 122 that delays dissolution of marker support 122. In one embodiment, the bioabsorbable coating may be formed from a polymeric material. Dissolution of the polymeric material may degrade over approximately two to four weeks, at which point the biodegradable marker support 122 would be exposed. The material of marker support 122 would then continue to degrade over the next two to four weeks, such that a total of approximately four to eight weeks passes before marker 130 is potentially unsupported. As previously mentioned, approximately three to six weeks is sufficient to allow for endothelialization to occur, and thus marker 130 will be part of the vessel wall once both the polymeric coating and the material of marker support 122 is absorbed by the body. The bioabsorbable polymeric material may include polymers or copolymers such as polylactide [poly-L-lactide (PLLA), poly-D-lactide (PDLA)], polyglycolide, polydioxanone, polycaprolactone, polygluconate, polylactic acid-polyethylene oxide copolymers, modified cellulose, collagen, poly(hydroxybutyrate), polyanhydride, polyphosphoester, poly(amino acids), poly(alpha-hydroxy acid) or related copolymers materials. The dissolution rate of the coating may be tailored by controlling the type of bioabsorbable polymer, the thickness and/or density of the bioabsorbable polymer, and/or the nature of the bioabsorbable polymer. For example, each type of bioabsorbable polymer has a characteristic degradation rate in the body. Some materials are relatively fast-bioabsorbing materials (weeks to months) while others are relatively slow-bioabsorbing materials (months to years). In addition, increasing thickness and/or density of a polymeric material will generally slow the dissolution rate of the coating. Characteristics such as the chemical composition and molecular weight of the bioabsorbable polymer may also be selected in order to control the dissolution rate of the coating.

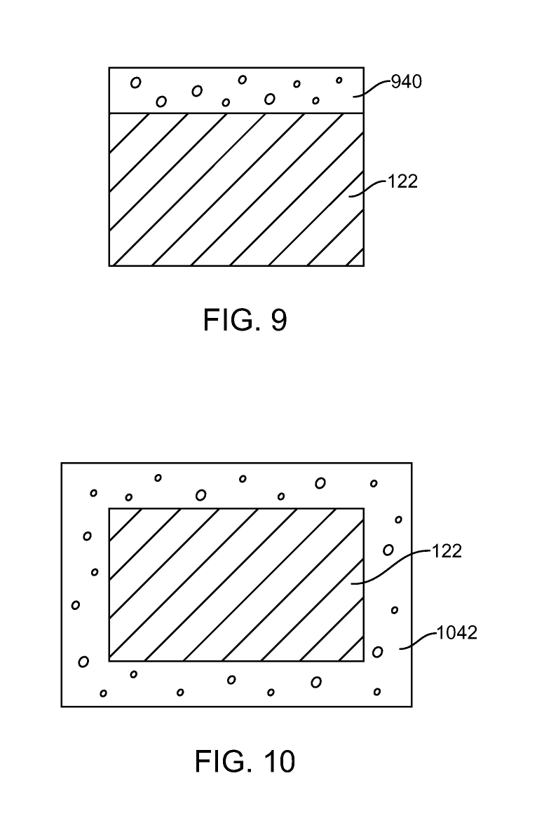

The coating may be applied to one or more surfaces of marker support 122 in order to isolate one or more body fluid contacting surfaces of marker support 122. For example, as shown in FIG. 9, a surface coating 940 is applied to one surface of marker support 122. FIG. 9 is a cross-sectional view of marker support 122 taken along line B-B of FIG. 2. When implanted into a target vessel, body fluid such as blood contacts stent 100 and acts as the corrosion agent. Preferably, surface coating 940 is applied to a select surface such that it prevents marker support 122 from coming into contact with a body fluid and therefore delays corrosion of marker support 122 until surface coating 940 is eroded. Surface coating 940 may additionally be applied to one or more body fluid contacting surfaces of marker 130. Accordingly, dissolution of the biodegradable material of marker support 122 is controlled or slowed so that marker support 122 will remain intact for a sufficient time to allow for marker 130 to endothelialize.

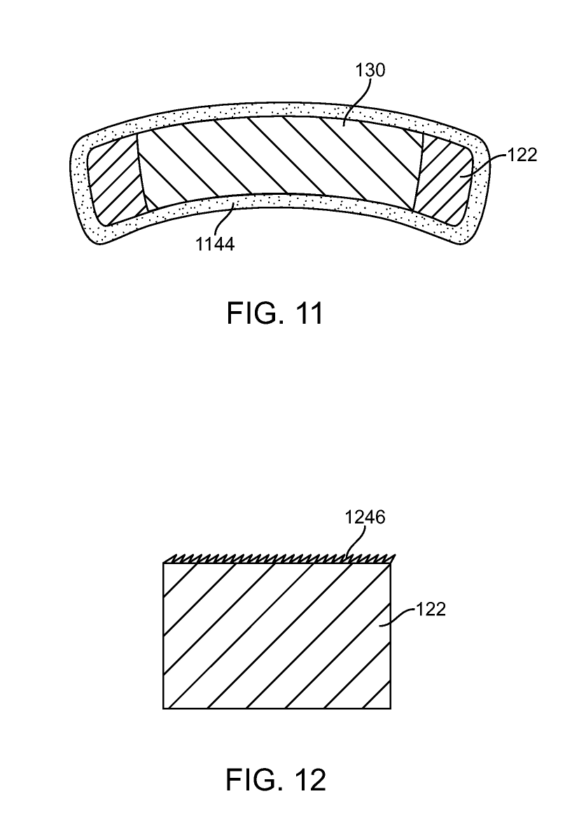

In another embodiment of the present invention, an encapsulating coating 1042 may also be utilized on marker support 122 as the dissolution control mechanism. For example, as shown in FIG. 10, encapsulating coating 1042 may be applied to all surfaces of marker support 122 in order to isolate marker support 122. FIG. 10 is a cross-sectional view of marker support 122 taken along line B-B of FIG. 2. Encapsulating coating 1042 prevents marker support 122 from coming into contact with a body fluid, and thus delays corrosion of marker support 122 until encapsulating coating 1042 is eroded. Further as shown in FIG. 11, an encapsulating coating 1144 may be applied to marker assembly 120 such that the coating material encapsulates both marker support 122 and marker 130. FIG. 11 is a cross-sectional view of marker assembly 120, including marker support 122 having marker 130 located therein, taken along line C-C of FIG. 3. An encapsulating coating, such as encapsulating coating 1042 or encapsulating coating 1144, will control or slow down dissolution of the biodegradable material of marker support 122 so that marker support 122 will remain intact for a sufficient time to allow for marker 130 to endothelialize.

In another embodiment of the present invention illustrated in FIG. 12, the dissolution control mechanism includes passivating or oxidizing one or more body fluid contacting surfaces of marker support 122. Passivating or oxidizing marker support 122 forms a protective corrosion-inhibiting barrier or layer 1246 that prevents premature dissolution of marker support 122. For example, as shown in FIG. 12, protective corrosion-inhibiting barrier or layer 1246 is formed on one surface of marker support 122. FIG. 12 is a cross-sectional view of marker support 122 taken along line B-B of FIG. 2. When implanted into a target vessel, body fluid such as blood contacts stent 100 and acts as the corrosion agent. Preferably, protective corrosion-inhibiting barrier or layer 1246 is formed to a select surface such that it delays corrosion of marker support 122 until protective corrosion-inhibiting barrier or layer 1246 is eroded. Accordingly, dissolution of the biodegradable material of marker support 122 is controlled or slowed so that marker support 122 will remain intact for a sufficient time to allow for marker 130 to endothelialize. For example, when marker support 122 is formed from magnesium, a solution of nitric oxide, chromic acid, or hydrofluoric (HF) acid may be utilized to oxidize material of marker support 122. In addition, electropolishing techniques may also be utilized to passivate or oxidize marker support 122.

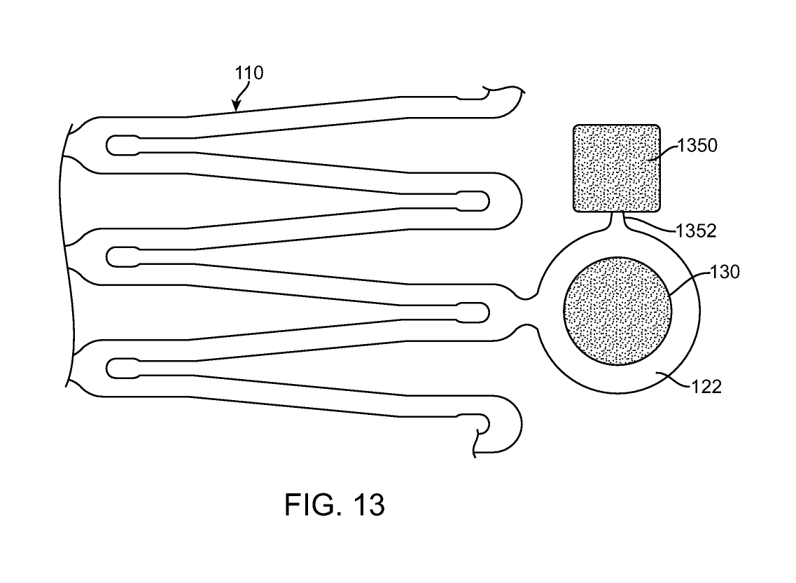

In another embodiment of the present invention illustrated in FIG. 13, the dissolution control mechanism is anodically protecting marker support 122 with a sacrificial anode 1350 having a higher corrosion potential than marker support 122. Sacrificial anode 1350 is electrically connected to marker support 122 so that an electrical pathway occurs between sacrificial anode 1350 and marker support 122. Due to the higher corrosion potential of sacrificial anode 1350, electrolytic galvanic corrosion of marker support 122 is redirected to sacrificial anode 1350, away from marker support 122, such that dissolution of the material of the marker support is delayed for a sufficient time for marker 130 to endothelialize. Therefore, the use of designated sacrificial anode 1350 will control or slow down dissolution of the biodegradable material of marker support 122 so that marker support 122 will remain intact for a sufficient time to allow for marker 130 to endothelialize. Thus, the amount of sacrificial anode 1350 utilized should preferably be sufficient to remain intact for a desired duration of time to permit complete endothelialization of marker 130.

As shown in the embodiment of FIG. 13, the sacrificial anode 1350 may be in the form of a tab adjacent marker support 122 which is electrically connected to marker support 122 by a tether or connector 1352. One or more sacrificial anode 1350 may be provided. More generally, one or more sacrificial anode portions may be provided at one or both ends of the stent or at any other suitable location in the stent as long as the sacrificial anode portions are electrically connected to marker support 122. In another embodiment, the sacrificial anode 1350 may be in the form of metal plating or a coating on a portion of marker support 122 or affixed, attached, or otherwise electrically connected to marker support 122. As apparent to one of ordinary skill in the art, in order for sacrificial anode 1350 to be electrically connected to marker support 122, it is not required that sacrificial anode 1350 be mechanically connected to marker support 122. For example, a body fluid such as blood may act to electrically connect sacrificial anode 1350 to marker support 122 without a mechanical connection between the two structures.

Any suitable material may be selected for sacrificial anode 1350 so long as the material has a higher corrosion potential than marker support 122. Materials utilized for the sacrificial anode 1350 may include but are not limited to magnesium or a magnesium alloy, zinc or a zinc alloy, a beryllium alloy, a lithium alloy, or an alloy containing two or more or the previously mentioned elements. The material selected for the sacrificial anode 1350 functions as an anode with respect to marker support 122 while the material for marker support 122 is, in turn, a cathode with respect to the sacrificial anode 1350. For example, sacrificial anode 1350 may be formed from 100% magnesium while marker support 122 is formed from a magnesium alloy.

Body portion 106 of stent 100 may be formed using any of a number of different methods. For example, body portion 106 may be formed by winding a wire or ribbon around a mandrel to form a strut pattern like those described above and then welding or otherwise mechanically connecting two ends thereof to form a cylindrical ring 108. A plurality of cylindrical rings 108 are subsequently connected together to form body portion 106. Alternatively, body portion 106 may be manufactured by machining tubing or solid stock material into toroid bands, and then bending the bands on a mandrel to form the pattern described above. A plurality of cylindrical rings formed in this manner are subsequently connected together to form the longitudinal stent body. Laser or chemical etching or another method of cutting a desired shape out of a solid stock material or tubing may also be used to form body portion 106 of the present invention. In this manner, a plurality of cylindrical rings may be formed connected together such that the stent body is a unitary structure. Further, body portion 106 of the present invention may be manufactured in any other method that would be apparent to one skilled in the art. The cross-sectional shape of stent 100 may be circular, ellipsoidal, rectangular, hexagonal rectangular, square, or other polygon, although at present it is believed that circular or ellipsoidal may be preferable.

Preferably, stent 100 is formed in an expanded state, crimped onto a conventional balloon dilation catheter for delivery to a treatment site and expanded by the radial force of the balloon. Conventional balloon catheters that may be used in the present invention includes any type of catheter known in the art, including over-the-wire catheters, rapid-exchange catheters, core wire catheters, and any other appropriate balloon catheters. For example, conventional balloon catheters such as those shown or described in U.S. Pat. Nos. 6,736,827; 6,554,795; 6,500,147; and 5,458,639, which are incorporated by reference herein in their entirety, may be used within the stent delivery catheter of the present invention.

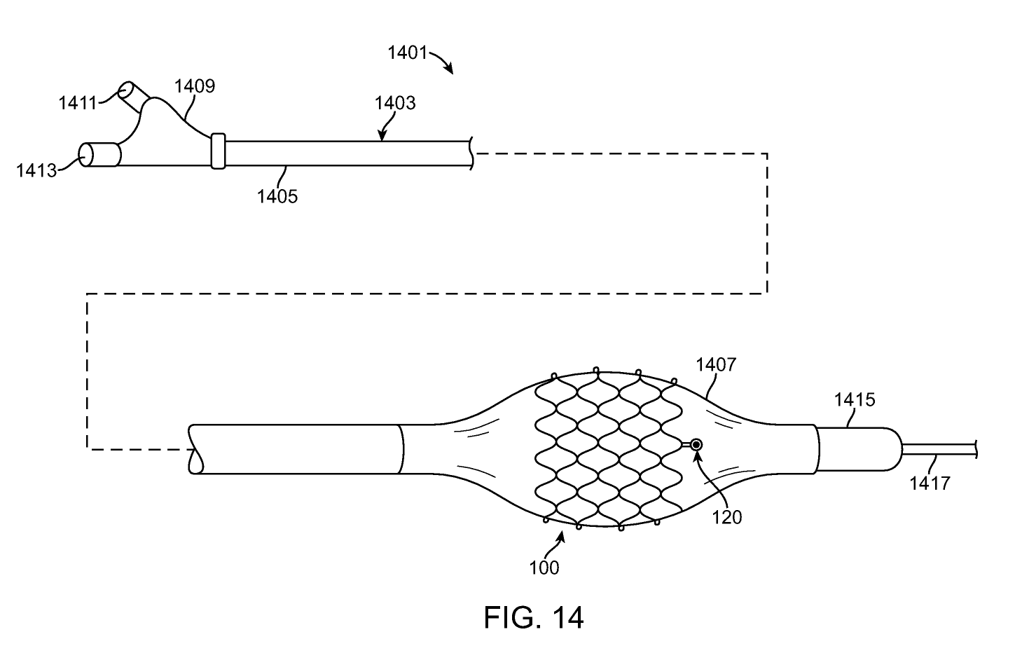

For example, FIG. 14 is an illustration of a stent delivery system 1401 for tracking stent 100 to the target site in accordance with an embodiment of the present invention. Stent delivery system 1401 includes a catheter 1403 having a proximal shaft 1405, a guidewire shaft 1415, and a balloon 1407. Proximal shaft 1405 has a proximal end attached to a hub 1409 and a distal end attached to a proximal end of balloon 1407. Guidewire shaft 1415 extends between hub 1409 and a distal tip of catheter 1403 through proximal shaft 1405 and balloon 1407. Hub 1409 includes an inflation port 1411 for coupling to a source of inflation fluid. Inflation port 1411 fluidly communicates with balloon 1407 via an inflation lumen (not shown) that extends through proximal shaft 1405. In addition, hub 1409 includes a guidewire port 1413 that communicates with a guidewire lumen (not shown) of guidewire shaft 1415 for receiving a guidewire 1417 there through. As described herein, guidewire shaft 1415 extends the entire length of catheter 1403 in an over-the-wire configuration. However, as would be understood by one of ordinary skill in the art, guidewire shaft 1415 may alternately extend only within the distal portion of catheter 1403 in a rapid-exchange configuration. A stent 100 having at least one marker assembly 120 attached thereto formed in accordance with an embodiment of the present invention is positioned over balloon 1407. If desired, a sheath (not shown) may be provided to surround stent 100 to facilitate tracking of the stent delivery system 1401 over guidewire 1417 through the vasculature to a site of a stenotic lesion.

Deployment of balloon expandable stent 100 is accomplished by tracking catheter 1403 through the vascular system of the patient until stent 100 is located within a target vessel. The treatment site may include target tissue, for example, a lesion which may include plaque obstructing the flow of blood through the target vessel. Once positioned, a source of inflation fluid is connected to inflation port 1411 of hub 1409 so that balloon 1407 may be inflated to expand stent 100 as is known to one of ordinary skill in the art. Balloon 1407 of catheter 1403 is inflated to an extent such that stent 100 is expanded or deployed against the vascular wall of the target vessel to maintain the opening. Stent deployment can be performed following treatments such as angioplasty, or during initial balloon dilation of the treatment site, which is referred to as primary stenting.

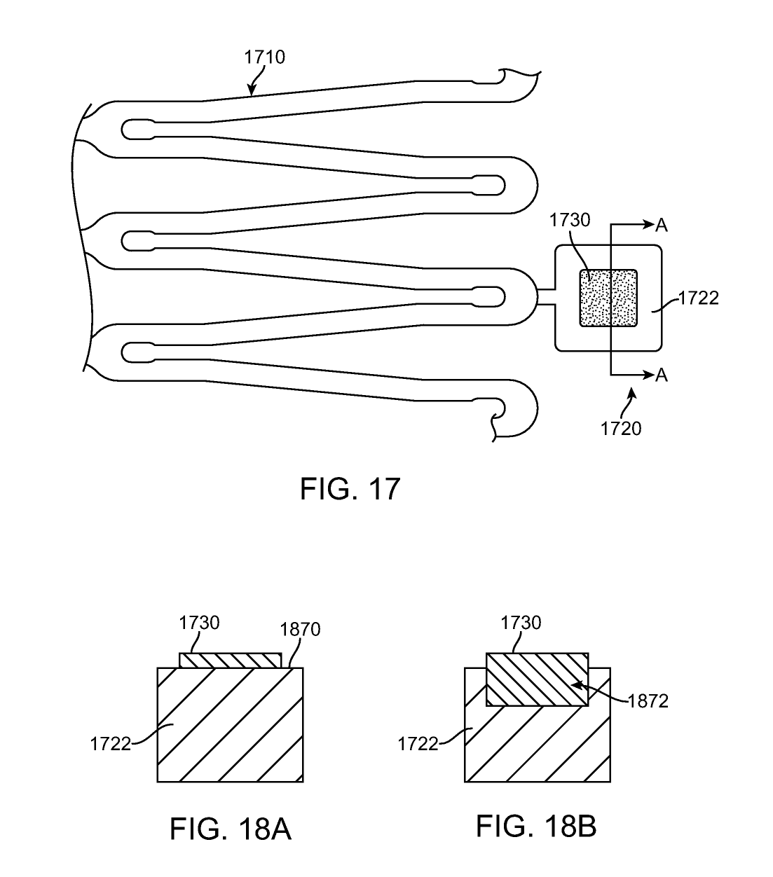

As will be apparent to those of ordinary skill in the art, rather than being disposed within an inner volume of an annular or ring shaped marker support, the marker may be disposed on a flat, tab-like marker support. As illustrated in FIG. 17, 18A, and 18B, marker 1730 may be in the form of a metal plate or band on a portion of a marker support 1722 or affixed, attached, or otherwise engaged to marker support 1722. Marker support 1722 has a flat, tab-like structure and may have any suitable shape including circular or rectangular. Marker 1730 may be affixed to marker support 1722 via the use of adhesives, laser welding techniques or other welding techniques or swaged onto marker support 1722. Marker 1730 may be disposed on an outer surface 1870 of marker support 1722, as shown in FIG. 18A. In another embodiment, marker 1730 may be disposed within a recess 1872 of marker support 1722 as shown in FIG. 18B. Endothelialization of marker 1730 disposed in recess 1872 would occur due to tissue ingrowth into and through marker 1730 such that marker 1730 may become incorporated into the walls of the lumen, including, for example, when marker 1730 has a relatively porous outer surface or contains protrusions or indentations on the outer surface in order to facilitate tissue ingrowth. The controlled dissolution of marker support 1722 may be accomplished via various mechanisms discussed in more detail above, including increasing the cross-sectional thickness of marker support 1722 relative to the cross-sectional thickness of stent strut 1710, utilizing a different, slower absorbing material for marker support 1722 relative to stent strut 1710, passivating or oxidizing marker support 1722, utilizing a bioabsorbable polymeric coating on marker support 1722, anodically protecting marker support 1722 with a sacrificial anode, or any other suitable means of slowing absorption or corrosion of marker support 1722.

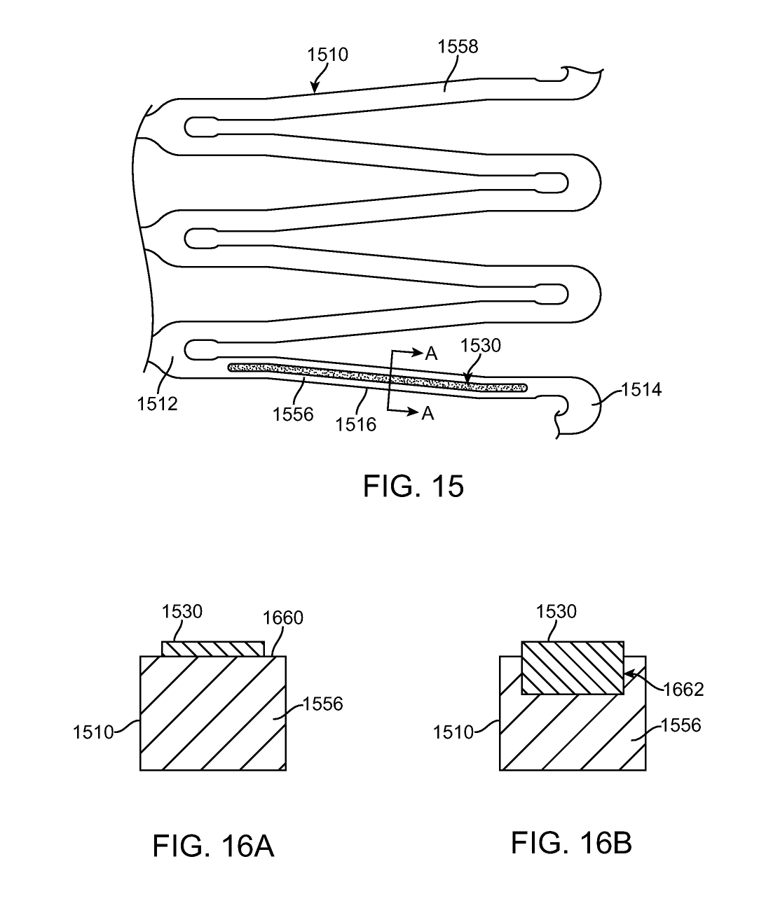

In addition, as will be apparent to those of ordinary skill in the art, rather than being adjacent to a body portion of the stent, the marker support may be formed integrally with the body portion. For example, the radiopaque marker may be disposed on or within a stent strut of the body portion. In other words, as illustrated in FIG. 15, 16A, and 16B, stent strut 1510 includes a first or marker support portion 1556 and a second or remaining portion 1558. The marker support is formed integrally with the first portion 1556 of stent strut 1510 and dissolution of the integral marker support/first portion 1556 is selectively controlled to biodegrade slower than the second portion 1558 of stent strut 1510 in order to allow time for marker 1530 to endothelialize. Marker 1530 may be in the form of a metal plate or band that is affixed, attached, or otherwise engaged to stent strut 1510. In FIG. 15, marker 1530 is affixed to a straight portion 1516 but may alternatively or additionally be affixed to peaks 1512 and/or valleys 1514. Further, marker 1530 is shown as a straight metal plate or band attached to stent strut 1510. However, marker 1530 may be any shape, such as circular, rectangular, oval, or a curvy strip. In addition, marker 1530 may be a metal plate or band wound or wrapped around the outer surface of stent strut 1510 in a helical fashion. Marker 1530 may be affixed to stent strut 1510 via the use of adhesives, laser welding techniques or other welding techniques or swaged onto stent strut 1510. Marker 1530 may be disposed on an outer surface 1660 of stent strut 1510, as shown in FIG. 16A. In another embodiment, marker 1530 may be fully or partially disposed within a recess 1662 of stent strut 1510 as shown in FIG. 16B. Endothelialization of marker 1530 disposed in recess 1662 would occur due to tissue ingrowth into and through marker 1530 such that marker 1530 may become incorporated into the walls of the lumen, including, for example, when marker 1530 has a relatively porous outer surface or contains protrusions or indentations on the outer surface in order to facilitate tissue ingrowth. When the marker support is formed integrally with first portion 1556 of stent strut 1510, controlled dissolution of the integral marker support/first portion 1556 may be accomplished via various mechanisms, including increasing the cross-sectional thickness of the integral marker support/first portion 1556 of the stent strut securing the marker versus the thickness of the second portion 1558 of the stent strut, utilizing a different, slower absorbing material for the integral marker support/first portion 1556 of the stent strut securing the marker, passivating or oxidation the integral marker support/first portion 1556 of the stent strut securing the marker, utilizing a bioabsorbable polymeric coating on the integral marker support/first portion 1556 of the stent strut securing the marker, and/or anodically protecting the integral marker support/first portion 1556 of the stent strut securing the marker with a sacrificial anode, as described in more detail above.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. An intraluminal stent device, comprising:
a biodegradable body portion having a proximal end, a distal end, and a generally cylindrical hollow shape, wherein the body portion has a first thickness;
at least one biodegradable marker support having a second thickness; and
a radiopaque marker attached to the marker support,
wherein the second thickness is greater than the first thickness so that upon implantation of the stent within the vasculature dissolution of the marker support is selectively controlled to biodegrade slower than the body portion of the stent in order to allow the marker to endothelialize.

2. The intraluminal stent device of claim 1, wherein the marker support biodegrades between 30-100% slower than the body portion of the stent.

3. The intraluminal stent device of claim 1, wherein the body portion and marker support are of a biodegradable material selected from a group consisting of magnesium and a magnesium alloy and the radiopaque marker is formed from tantalum.

4. The intraluminal stent device of claim 1, wherein an outer surface of the radiopaque marker includes an irregular surface in order to facilitate endothelialization of the radiopaque marker, the irregular surface being selected from a group consisting of a surface including protrusions thereon, a surface including indentations thereon, and a relatively porous surface.

5. The intraluminal stent device of claim 1, wherein the at least one marker support has a configuration selected from the group consisting of an annular shape or a flat tab and is connected to one of the proximal end and the distal end of the body portion.

6. The intraluminal stent device of claim 1, wherein the marker support is an integral portion of the body portion of the stent.

7. An intraluminal stent device, comprising:
a biodegradable body portion having a proximal end, a distal end, and a generally cylindrical hollow shape;
at least one biodegradable marker support;
a radiopaque marker attached to the marker support; and
a bioabsorbable coating placed over at least a portion of the marker support so that upon implantation of the stent within the vasculature dissolution of the marker support is selectively controlled to biodegrade slower than the body portion of the stent in order to allow the marker to endothelialize.

8. The intraluminal stent device of claim 7, wherein the coating is a polymeric coating.

9. The intraluminal stent device of claim 9, wherein the coating is encapsulates the marker support.

10. The intraluminal stent device of claim 9, wherein the coating is placed over a surface of the marker support to isolate the marker support from contacting a body fluid.

11. The intraluminal stent device of claim 7, wherein the body portion and marker support are of a biodegradable material selected from a group consisting of magnesium and a magnesium alloy and the radiopaque marker is formed from tantalum.

12. The intraluminal stent device of claim 7, wherein an outer surface of the radiopaque marker includes an irregular surface in order to facilitate endothelialization of the radiopaque marker, the irregular surface being selected from a group consisting of a surface including protrusions thereon, a surface including indentations thereon, and a relatively porous surface.

13. The intraluminal stent device of claim 7, wherein the at least one marker support has a configuration selected from the group consisting of an annular shape or a flat tab and is connected to one of the proximal end and the distal end of the body portion.

14. The intraluminal stent device of claim 7, wherein the marker support is an integral body of the body portion of the stent.

15. An intraluminal stent device, comprising:
a biodegradable body portion having a proximal end, a distal end, and a generally cylindrical hollow shape;
at least one biodegradable marker support;
a radiopaque marker attached to the marker support; and
a corrosion-resistant layer formed by oxidizing or passivating at least a portion of the marker support so that upon implantation of the stent within the vasculature dissolution of the marker support is selectively controlled to biodegrade slower than the body portion of the stent in order to allow the marker to endothelialize.

16. The intraluminal stent device of claim 15, wherein the body portion and marker support are of a biodegradable material selected from a group consisting of magnesium and a magnesium alloy and the radiopaque marker is formed from tantalum.

17. The intraluminal stent device of claim 15, wherein an outer surface of the radiopaque marker includes an irregular surface in order to facilitate endothelialization of the radiopaque marker, the irregular surface being selected from a group consisting of a surface including protrusions thereon, a surface including indentations thereon, and a relatively porous surface.

18. The intraluminal stent device of claim 15, wherein the at least one marker support has a configuration selected from the group consisting of an annular shape or a flat tab and is connected to one of the proximal end and the distal end of the body portion.

19. The intraluminal stent device of claim 15, wherein the marker support is an integral portion of the body portion of the stent.

20. An intraluminal stent device, comprising:
a biodegradable body portion having a proximal end, a distal end, and a generally cylindrical hollow shape;
at least one biodegradable marker support formed of a first biodegradable material having a first corrosion potential;
a radiopaque marker attached to the marker support; and
a sacrificial anode electrically connected to the marker support, wherein the sacrificial anode is formed of a second biodegradable material having a second corrosion potential that is higher than the first corrosion potential of the marker support so that upon implantation of the stent within the vasculature dissolution of the marker support is selectively controlled to biodegrade slower than the body portion of the stent in order to allow the marker to endothelialize.

21. The intraluminal stent device of claim 20, wherein the body portion and marker support are of a biodegradable material selected from a group consisting of magnesium and a magnesium alloy and the radiopaque marker is formed from tantalum.

22. The intraluminal stent device of claim 20, wherein an outer surface of the radiopaque marker includes an irregular surface in order to facilitate endothelialization of the radiopaque marker, the irregular surface being selected from a group consisting of a surface including protrusions thereon, a surface including indentations thereon, and a relatively porous surface.

23. The intraluminal stent device of claim 20, wherein the at least one marker support has a configuration selected from the group consisting of an annular shape or a flat tab and is connected to one of the proximal end and the distal end of the body portion.

24. The intraluminal stent device of claim 20, wherein the marker support is an integral portion of the body portion of the stent.

25. An intraluminal stent device, comprising:
a biodegradable body portion having a proximal end, a distal end, and a generally cylindrical hollow shape, wherein the body portion is formed of a first biodegradable material having a first dissolution rate;
at least one biodegradable marker support formed of a second biodegradable material having a second dissolution rate; and
a radiopaque marker attached to the marker support,
wherein the second dissolution rate is slower than the first dissolution rate so that upon implantation of the stent within the vasculature dissolution of the marker support is selectively controlled to biodegrade slower than the body portion of the stent in order to allow the marker to endothelialize.
